(19) United States
(12) Patent Application Publication
(10) Pub. No.: US 2022/0356471 A1
(43) Pub. Date: Nov. 10, 2022

(54) CORRECTION OF EXON SKIPPING IN MONOCYTE-DERIVED CELLS FOR IMPROVED IMMUNE RESPONSE

(71) Applicant: D. Christopher Schelling, Bend, OR (US)

(72) Inventor: D. Christopher Schelling, Bend, OR (US)

(21) Appl. No.: 17/738,529

(22) Filed: May 6, 2022

Related U.S. Application Data
(60) Provisional application No. 63/185,980, filed on May 7, 2021.
(60) Provisional application No. 63/333,339, filed on Apr. 21, 2022.

(51) Int. Cl. C12N 15/113; A61K 45/06

(52) U.S. Cl. CPC C12N 15/113; A61K 45/06; C12N 2320/33; C12N 2310/11; C12N 2320/32

(57) ABSTRACT

The present disclosure relates generally to compositions and methods for inhibiting exon 3 skipping in the 6-pyruvoyltetrahydropterin synthase (PTS) gene in monocytes, monocyte-derived cells such as macrophages and dendritic cells of the precursor cells thereof. The inhibition can be achieved with genome editing of the genomic sequence to remove certain splicing factor recognition sites or inhibiting the expression or activity of the splicing factors that contribute to the cell-specific exon skipping. Monocytes and monocyte-derived cells that have reduced exon skipping in the PTS gene can generate more potent immune response and thus are useful in preventing or treating diseases such as infectious diseases and cancer.

### CROSS REFERENCE TO RELATED APPLICATIONS

This application claims the benefit under 35 U.S.C. § 119(e) of the U.S. Provisional Application Serial Nos. 63/185,980, filed May 7, 2021 and 63/333,339, filed Apr. 21, 2022, the content of each of which is hereby incorporated by reference in its entirety.

### SEQUENCE LISTING

The instant application contains a Sequence Listing which has been submitted in ASCII format via EFS-Web and is hereby incorporated by reference in its entirety. Said ASCII copy, created on May 3, 2022, is named 334203.txt and is 10,310 bytes in size.

### BACKGROUND

The immune system is a host defense system that protects against diseases. The immune system can detect a wide variety of agents, known as pathogens such as viruses, bacteria, and parasitic worms, and distinguish them from the organism's own healthy tissue. Pathogens can evolve and adapt, and thereby avoid detection and neutralization by the immune system. Jawed vertebrates, including humans, meanwhile, have developed sophisticated mechanisms to adapt to such changes.

Disorders of the immune system can result in autoimmune diseases, inflammatory diseases and cancer. Immunodeficiency occurs when the immune system is less active than normal, resulting in recurring and life-threatening infections. In humans, immunodeficiency can either be the result of a genetic disease such as severe combined immunodeficiency, acquired conditions such as HIV/AIDS, or the use of immunosuppressive medication. Boosting the immune system, therefore, can be useful in ameliorating immunodeficiency and treating infectious diseases.

Cancer immunotherapy is the artificial stimulation of the immune system to treat cancer, improving on the system's natural ability to fight cancer. Cancer cells often have tumor antigens, which can be detected by antibodies capable of recognizing these antigens. The tumor antigens are often proteins or other macromolecules such as carbohydrates. Once detected, the cancer cells may be killed by mechanisms such as antibody-dependent cellular cytotoxicity (ADCC).

### SUMMARY

The present disclosure, in various embodiments, describes compositions and methods for inhibiting and/or correcting exon 3 skipping in the 6-pyruvoyltetrahydropterin synthase (PTS) gene in human monocytes, monocyte-derived cells (e.g., macrophages and dendritic cells), or their precursor cells. The inhibition can be achieved with editing of the genomic sequence of PTS to alter or remove certain splicing factor recognition sites or inhibiting the expression or activity of the splicing factors that contribute to the cell type-specific exon skipping. Human monocytes and monocyte-derived cells generally have no detectable normal protein product of the PTS gene (the PTPS protein) as most of the transcripts in these cells produce an inactive protein due to cell type-specific alternative splicing that skips exon 3. Restoring the PTPS protein activity in these cells, therefore, can strengthen the immune system of the human subject, and help prevent and treat diseases such as infectious diseases, cancer, cardiovascular diseases, cerebrovascular diseases, autoimmune diseases, and liver disorders.

One embodiment of the present disclosure provides a method for altering the immune system in a human subject in need thereof, comprising editing the 6-pyruvoyltetrahydropterin synthase (PTS) genomic sequence or pre-mRNA in a monocyte, a macrophage, a dendritic cell or a precursor cell thereof in the human subject, wherein the editing inhibits skipping of exon 3 in the PTS mRNA during splicing.

In some embodiments, the editing alters a recognition site of a splicing factor. In some embodiments, the recognition site is located within intron 2 or intron 3. In some embodiments, the recognition site is located within intron 2. In some embodiments, the splicing factor is selected from the group consisting of SRSF1, SRSF2, SRSF3, SRSF4, SRSF5, SRSF6, SRSF7, SRSF8, SRSF9, SRSF10, SRSF11 and SRSF12. In some embodiments, the recognition site is selected from Table 3.

In some embodiments, the splicing factor is SRSF3. In some embodiments, the recognition site is CTCTTCC, corresponding to nucleotides 2895-2901 of SEQ ID NO:1.

In some embodiments, the splicing factor is SRSF1. In some embodiments, the recognition site is selected from the group consisting of TGGTGGC (2833-2839 of SEQ ID NO:1), TGATGCT (3541-3547 of SEQ ID NO:1), TGGTGGA (3927-3933 of SEQ ID NO:1), TGGTGCT (4105-4111 of SEQ ID NO:1), and TGAAGGC (4143-4149 of SEQ ID NO:1).

In some embodiments, the editing comprises addition, deletion and/or substitution of at least one, two, three or four of the nucleotides in the recognition site. In some embodiments, the edited recognition site is not capable of being bound by the respective splicing factor.

In some embodiments, the editing comprises introducing to the cell an editing system targeting the recognition site. In some embodiments, the editing system is selected from a CRISPR/Cas (Clustered regularly interspaced short palindromic repeats/CRISPR-associated protein) system, a gene editor comprising a CRISPR/Cas system with a cytosine deaminase, a gene editing system based on a meganuclease, a zinc finger nuclease (ZFN), or a transcription-activator like effector nucleases (TALEN).

In some embodiments, the human subject suffers from an infectious disease or condition, or cancer or is at risk of developing an infectious disease or condition, cancer, a cardiovascular disease, a cerebrovascular disease, an autoimmune disease, or a liver disorder.

Also provided, in one embodiment, is a method for modulating or altering the immune system in a human subject in need thereof, comprising administering to the human subject a human monocyte, macrophage, dendritic cell or a precursor cell thereof in which the 6-pyruvoyltetrahydropterin synthase (PTS) genomic sequence has been edited to inhibit skipping of exon 3 in the PTS mRNA during splicing.

In some embodiments, the monocyte, macrophage, dendritic cell or the precursor cell thereof has been retrieved from the human subject and edited in vitro or ex vivo. In some embodiments, the monocyte or macrophage has been dedifferentiated prior to administration.

In some embodiments, the editing alters a recognition site of a splicing factor. In some embodiments, the splicing factor is selected from the group consisting of SRSF1, SRSF2, SRSF3, SRSF4, SRSF5, SRSF6, SRSF7, SRSF8, SRSF9, SRSF10, SRSF11 and SRSF12. In some embodiments, the recognition site is CTCTTCC, corresponding to nucleotides 2895-2901 of SEQ ID NO:1.

Also provided, in one embodiment, is a method for altering the immune system in a human subject in need thereof, comprising administering to the subject an agent that inhibits exon 3 skipping or promotes exon 3 recognition/inclusion in the 6-pyruvoyltetrahydropterin synthase (PTS) pre-mRNA in a monocyte, a macrophage, a dendritic cell or a precursor cell thereof in the subject.

In some embodiments, the agent inhibits the biological activity of a splicing factor selected from the group consisting of SRSF1, SRSF2, SRSF3, SRSF4, SRSF5, SRSF6, SRSF7, SRSF8, SRSF9, SRSF10, SRSF11 and SRSF12. In some embodiments, the splicing factor is SRSF3. In some embodiments, the agent is an antisense oligonucleotide or RNA, a siRNA, or a shRNA that inhibits the expression of the splicing factor. In some embodiments, the agent is an antibody having specificity to the splicing factor.

In some embodiments, the agent is a small molecule agent selected from the group consisting of kinetin, epigallocatechin gallate (EGCG), genistein, daidzein, a cardiac glycoside, and rectifier of aberrant splicing (RECTAS). In some embodiments, the cardiac glycoside is selected from the group consisting of digoxin digitonin, digitoxin, digoxigenin, digitoxigenin, acetyldigitoxin, bufalin, ouabagenin and ouabain.

In some embodiments, the agent that promotes exon 3 recognition or inclusion is a U1snRNA that recognizes exon 3 in the PTS pre-mRNA.

Also provided, in one embodiment, is a method for altering the immune system in a mammalian subject in need thereof, comprising administering a nonsense suppression agent to a tissue in the subject that produces or circulates monocytes, macrophages, dendritic cells or precursor cells thereof.

In some embodiments, the nonsense suppression agent is selected from the group consisting of a stop codon readthrough drug, a suppressor tRNA, a stop codon pseudouridylation agent, and a nonsense-mediated mRNA decay (NMD) inhibitor. In some embodiments, the nonsense suppression agent is selected from the group consisting of ataluren, an aminoglycoside, gentamicin, amikacin, negamycin, spiramycin, josamycin, tylosin and amlexanox.

In some embodiments, the tissue is bone marrow.

In some embodiments, the administration is targeted at the monocyte, macrophage, dendritic cell or the precursor cell thereof. In some embodiments, the subject suffers from an infectious disease or condition, or cancer or is at risk of developing an infectious disease or condition, or cancer.

In some embodiments, the subject has increased immune activity as compared to a healthy individual. In some embodiments, the increased immune activity is measured with a biomarker from a sample selected from the group consisting of serum, urine, cerebrospinal fluid, synovial fluid, saliva, ascitic fluid, bile, pancreatic juice and gastric juice. In some embodiments, the biomarker is selected from the group consisting of neopterin, C-reactive protein, interferon-gamma, interferon-alpha, interferon-beta, and procalcitonin.

### DETAILED DESCRIPTION

The following description sets forth exemplary embodiments of the present technology. It should be recognized, however, that such description is not intended as a limitation on the scope of the present disclosure but is instead provided as a description of exemplary embodiments.

### Activation of PTPS-Deficient Monocytes

PTPS (6-pyruvoyltetrahydropterin synthase, EC 4.2.3.12) is an enzyme that catalyzes the biosynthesis of 6-pyruvoyltetrahydropterin from GTP, which is used as a cofactor in the synthesis of aromatic amino acid monooxygenases and nitric oxide (NO) synthase. PTPS is a hexamer composed of identical subunits formed from a dimer of trimers. A 12-stranded antiparallel (3-barrel is formed by the trimer of dimers and creates a pore within PTPS. An enzymatic active site of PTPS is located where the three monomers come together in each subunit of the hexamer. PTPS is an intermediary in the synthetic pathway, or precursor, to tetrahydrobiopterin (BH4), a cofactor of several enzymes systems, including the aromatic amino acid monooxygenases (e.g., phenylalanine hydroxylase, tyrosine hydroxylase, tryptophan hydroxylase), the nitric oxide synthases (e.g., inducible nitric oxide synthase, endothelial nitric oxide synthase, neuronal nitric oxide synthase), and ether lipid oxidase.

The PTPS protein is encoded by the PTS gene in human. Mutations in the PTS gene have been suggested as the cause of a hereditary dystonic disorder. Four such mutations have been found, two of which are homozygous mutations, R25Q and I114V, and two heterozygous mutations, R16C and K120stop.

A lack of PTPS activity is a common cause of a deficiency of tetrahydrobiopterin. Tetrahydrobiopterin deficiency leads to hyperphenylalaninemia and the inability to make neurotransmitters such as dopamine and serotonin. PTPS deficiency has also been shown to lead to severe mental retardation, delayed motor development, and seizures. Low levels of tetrahydrobiopterin production, as opposed to near complete lack of tetrahydrobiopterin, may cause fluctuations in the symptoms.

A connection between the dysfunction of PTPS and immune deficiency or infectious diseases has not been established. Through analysis of its biochemical role in different cells and how it has evolved from animals to human, however, the instant inventor has determined that activation of PTPS in human monocytes and monocyte-derived cells (e.g., macrophages and dendritic cells) can boost immune response which can be helpful in preventing and treating infectious diseases and cancer.

Unlike other tissues such as the liver and the central nervous system, the human monocytes and monocyte-derived cells have almost no detectable PTPS activity. The transcription levels of the PTS gene in the monocytes and monocyte-derived cells are normal. Based on sequence analysis and mechanistic studies, the instant inventor contemplates that the reduced PTPS activity in human monocytes and monocyte-derived cells is due to exon 3 skipping, rather than posttranscriptional mechanisms. When the exon 3 is skipped, it results in a shortened transcript with a premature stop codon. Similar differential expression patterns are also observed in higher primates such as chimpanzees. Such exon skipping does not occur, or at least is not prevalent, in many other types of human cells, or in the monocytes and monocyte-derived cells of lower level animals such as rats.

Reduced PTPS activity in human monocytes and monocyte-derived cells may have its evolutionary advantages but is contemplated to contribute to the weakened immune system in human. Therefore, at least in human subjects where a stronger immune system is desired, such as in those suffering from or at risk of developing infectious diseases or cancer, reactivation of PTPS in monocytes or monocyte-derived cells can be advantageous. In accordance with one embodiment of the present disclosure, therefore, provided are compositions and methods for altering or increasing the activity of PTPS in human monocytes and monocyte-derived cells.

### Gene Editing

As demonstrated in Example 1, introns 2 and 3 of the PTS gene harbor a number of potential binding sites for splicing factors such as serine/arginine-rich splicing factor 1 (SRSF1) and serine/arginine-rich splicing factor 3 (SRSF3) (formerly known as SFRS1 and SFRS3, respectively). These splicing factor binding sites are contemplated to be involved in exon 3 skipping that leads to PTPS inactivation in human monocytes and monocyte-derived cells. Accordingly, if one or more of these binding sites is altered to inhibit binding by the respective splicing factor, exon 3 skipping can be reduced, leading to higher production of active PTPS proteins.

In some embodiments, accordingly, the present disclosure provides a method for altering or strengthening the immune system in a human subject in need thereof. The method can include editing the 6-pyruvoyltetrahydropterin synthase (PTS) genomic sequence or pre-mRNA in a monocyte, a macrophage, a dendritic cell or a precursor cell thereof in the human subject. In some embodiments, the editing inhibits skipping of exon 3 in the PTS mRNA during splicing. In some embodiments, the editing alters a recognition site of a splicing factor. In some embodiments, the recognition site is located within intron 2 or intron 3 of the gene.

Splicing factors are proteins involved in the removal of introns from strings of messenger RNA, so that the exons can bind together. Splicing factors may be part of complexes known as spliceosomes. The splicing reaction is carried out by the spliceosome, which consists of five small nuclear ribonucleoprotein complexes, or snRNPs, and as many as 50-100 non-snRNP proteins. Spliceosome assembly also requires SR proteins, a family of sequence specific splicing factors that have one or two RNA recognition motifs followed by an arginine/serine rich domain. Non-limiting examples of splicing factors include serine/arginine-rich splicing factor 1 (SRSF1), SRSF2, SRSF3, SRSF4, SRSF5, SRSF6, SRSF7, SRSF8, SRSF9, SRSF10, SRSF11 and SRSF12.

In one embodiment, at least one of the recognition sites in the PTS sequence is mutated. The mutation can be made within the genomic sequence of the host cell or introduced into the host cell as an extra copy. In some embodiments, the introduced sequence is a mutated genomic sequence. In some embodiments, the introduced sequence is the wild-type cDNA sequence or biological equivalents.

The mutation can be made at genomic or pre-mRNA level, without limitation. In some embodiments, the recognition site is recognized by any of SRSF1, SRSF2, SRSF3, SRSF4, SRSF5, SRSF6, SRSF7, SRSF8, SRSF9, SRSF10, SRSF11 and/or SRSF12.

In some embodiments, the recognition site is recognized by SRSF3. In some embodiments, the recognition site has the sequence of CTCTTCC (nucleotides 2895-2901 of SEQ ID NO:1), which is located in intron 2, 5′ of exon 3. The mutant can include one, two, three, four, five, six or seven substitutions, deletions, additions, of the combinations thereof. In some embodiments, 1, 2, 3, 4, 5, 6 or 7 of the nucleotides are deleted. In some embodiments, 1, 2, 3, 4, 5 or 6 additional nucleotides are inserted. In some embodiments, 1, 2, 3, 4, 5, 6 or 7 of the nucleotides are substituted, preferably with a nucleotide that does not fall into the consensus sequence (CUCKUCY).

In some embodiments, the SRSF3 recognition site is selected from the group consisting of TCAAC (2505-2509 of SEQ ID NO:1), ACAAC (2569-2573 of SEQ ID NO:1), TCTTC (2896-2900 of SEQ ID NO:1), ACTAC (3102-3106 of SEQ ID NO:1), TCTAC (3508-3512 of SEQ ID NO:1), TCTAC (3508-3512 of SEQ ID NO:1), TCTAC (3514-3518 of SEQ ID NO:1) and TCTAC (4026-4030 of SEQ ID NO:1). The mutant can include one, two, three, four, or five, substitutions, deletions, additions, of the combinations thereof. In some embodiments, 1, 2, 3, 4, or 5 of the nucleotides are deleted. In some embodiments, 1, 2, 3, 4, 5 or 6 additional nucleotides are inserted. In some embodiments, 1, 2, 3, 4, or 5 of the nucleotides are substituted, preferably with a nucleotide that does not fall into the consensus sequence (WCWWC).

In some embodiments, the splicing factor recognition site is recognized by SRSF1. In some embodiments, the splicing factor recognition site has a sequence selected from TGGTGGC (2833-2839 of SEQ ID NO:1), TGATGCT (3541-3547 of SEQ ID NO:1), TGGTGGA (3927-3933 of SEQ ID NO:1), TGGTGCT (4105-4111 of SEQ ID NO:1), or TGAAGGC (4143-4149 of SEQ ID NO:1). The mutant can include one, two, three, four, five, six or seven substitutions, deletions, additions, of the combinations thereof. In some embodiments, 1, 2, 3, 4, 5, 6 or 7 of the nucleotides are deleted. In some embodiments, 1, 2, 3, 4, 5 or 6 additional nucleotides are inserted. In some embodiments, 1, 2, 3, 4, 5, 6 or 7 of the nucleotides are substituted, preferably with a nucleotide that does not fall into the consensus sequence (UGRWGVH).

In some embodiments, the splicing factor recognition site is any one selected from Table 3. The mutant can include one, two, three, four, five, six or seven substitutions, deletions, additions, of the combinations thereof. In some embodiments, 1, 2, 3, 4, 5, 6 or 7 of the nucleotides are deleted. In some embodiments, 1, 2, 3, 4, 5 or 6 additional nucleotides are inserted. In some embodiments, 1, 2, 3, 4, 5, 6 or 7 of the nucleotides are substituted, preferably with a nucleotide that does not fall into the consensus sequence.

The mutation can be made in situ, by introducing to the target cell a genome editing system. Alternatively, a mutated sequence can be introduced to the target cell with a suitable vector, such as a viral vector. Methods for carrying out each of the procedures are known in the art.

Genome editing is a type of genetic engineering in which DNA is inserted, deleted or replaced in the genome of a living organism using engineered nucleases. A recently developed genome editing technology uses a CRISPR/Cas (Clustered regularly interspaced short palindromic repeats/CRISPR-associated protein) system. Directed by guide RNA (gRNA), a Cas nuclease can generate DNA double strand breaks (DSBs) at the targeted genomic sites in various cells (both cell lines and cells from living organisms). These DSBs are then repaired by the endogenous DNA repair system, which could be utilized to perform desired genome editing.

In general, two major DNA repair pathways could be activated by DSBs, non-homologous end joining (NHEJ) and homology-directed repair (HDR). NHEJ can introduce random insertions/deletions (indels) in the genomic DNA region around the DSBs, thereby leading to open reading frame (ORF) shift and ultimately gene inactivation. In contrast, when HDR is triggered, the genomic DNA sequence at target site could be replaced by the sequence of the exogenous donor DNA template through a homologous recombination mechanism, which can result in the correction of genetic mutation.

Base editors (BE), which integrate the CRISPR/Cas system with the APOBEC (apolipoprotein B mRNA editing enzyme, catalytic polypeptide-like) cytosine deaminase family, were later developed that greatly enhanced the efficiency of CRISPR/Cas9-meditated gene correction. Through fusion with Cas9 nickase (nCas9), the cytosine (C) deamination activity of rat APOBEC1 (rA1) can be purposely directed to the target bases in genome and to catalyze C to Thymine (T) substitutions at these bases.

In some embodiments, a Cas9 nickase is used in combination with guide sequence(s), e.g., two guide sequences, which target respectively sense and antisense strands of the DNA target. This combination allows both strands to be nicked and used to induce NHEJ. The ability of a candidate guide sequence to direct sequence-specific binding of a CRISPR complex to a target sequence may be assessed by any suitable assay. For example, the components of a CRISPR system sufficient to form a CRISPR complex, including the guide sequence to be tested, may be provided to a host cell having the corresponding target sequence, such as by transfection with vectors encoding the components of the CRISPR sequence, followed by an assessment of preferential cleavage within the target sequence. Similarly, cleavage of a target polynucleotide sequence may be evaluated in a test tube by providing the target sequence, components of a CRISPR complex, including the guide sequence to be tested and a control guide sequence different from the test guide sequence, and comparing binding or rate of cleavage at the target sequence between the test and control guide sequence reactions. In some embodiments, a guide sequence is selected to reduce the degree of secondary structure within the guide sequence. Secondary structure may be determined by any suitable polynucleotide folding algorithm.

A few other classes of nucleases can also generate DSBs useful for genome editing. They include meganucleases, the zinc finger nucleases (ZFNs), and transcription-activator like effector nucleases (TALEN).

Meganucleases are enzymes in the endonuclease family which are characterized by their capacity to recognize and cut large DNA sequences (from 14 to 40 base pairs). Meganucleases are found in microbial species, but it is difficult to find natural meganucleases for a specific DNA sequence. A large bank containing several tens of thousands of protein units, however, has been created. These units can be combined to obtain chimeric meganucleases that recognize the target sites.

Unlike meganucleases, ZFNs and TALEN use non-specific DNA cutting catalytic domains linked to specific DNA sequence recognizing peptides such as zinc fingers and transcription activator-like effectors (TALEs). The first step to this was to find an endonuclease whose DNA recognition site and cleaving site were separate from each other, a situation that is not the most common among restriction enzymes. Once this enzyme was found, its cleaving portion can be separated which would be very non-specific as it would have no recognition ability. This portion could then be linked to sequence recognizing peptides that can lead to high specificity. Zinc finger motifs occur in several transcription factors. In transcription factors, it is most often located at the protein-DNA interaction sites, where it stabilizes the motif. The C-terminal part of each finger is responsible for the specific recognition of the DNA sequence.

Transcription activator-like effector nucleases (TALENs) are specific DNA-binding proteins that feature an array of 33 or 34-amino acid repeats. TALENs are artificial restriction enzymes designed by fusing the DNA cutting domain of a nuclease to TALE domains, which can be tailored to specifically recognize a unique DNA sequence. These fusion proteins serve as readily targetable “DNA scissors” for gene editing applications. The DNA binding domains, which can be designed to bind any desired DNA sequence, comes from TAL effectors, DNA-binding proteins excreted by plant pathogenic Xanthomanos app. TAL effectors consist of repeated domains, each of which contains a highly considered sequence of 34 amino acids, and recognize a single DNA nucleotide within the target site. The nuclease can create double strand breaks at the target site that can be repaired by error-prone non-homologous end-joining (NHEJ), resulting in gene disruptions through the introduction of small insertions or deletions. These TALENs can be fused to the catalytic domain from a DNA nuclease, FokI, to generate a transcription activator-like effector nuclease (TALEN). The resultant TALEN constructs combine specificity and activity, effectively generating engineered sequence-specific nucleases that bind and cleave DNA sequences only at pre-selected sites.

The genome editing can take place in vivo, e.g., by administering the genome editing system (e.g., CRISPR/Cas and guide RNA) into the subject of interest. The administration can be systemic, but preferably targeted at the monocyte or monocyte-derived cells, or bone marrow cells or other precursor cells that can be differentiated into monocytes or monocyte-derived cells. Alternatively, in some embodiments, monocytes, monocyte-derived cells or their precursor cells can be introduced into the subject after the genome editing is completed in vitro or ex vivo in the cells.

Monocytes differentiate from hematopoietic stem cells, specifically granulocyte/macrophage progenitors in the bone marrow and enter the periphery as circulating monocytes. Various microenvironmental cues determine monocyte fate which can lead to differentiation into macrophage and dendritic cells. Monocytes are not simply macrophage and dendritic cell precursors but are also immune effector cells. Under inflammatory conditions, circulating monocytes can be recruited to the site of infection or injury, and once there, differentiate.

Liposomes are a commonly used delivery system for monocyte/phagocyte-targeted therapies providing advantages such as low immunogenicity, biocompatibility, cell specificity and drug protection. Parenterally administered liposomes are naturally cleared by the mononuclear phagocytic system (MPS). Liposome drug delivery systems take advantage of the physiological role of these cells to provide specific targeting and enhance delivery efficacy.

Targeting of liposomes to monocytes and monocyte-derived cells can be achieved by modifying lipid composition to control physicochemical properties such as size and charge and by the inclusion of surface ligands including proteins, peptides, antibodies, polysaccharides, glycolipids, glycoproteins, and lectins. Non-limiting examples of such ligands include anionic lipids, muramyl tripeptide (MTP), Arg-Gly-Asp (RGD), antibodies specific to VACM-1, CC52, CC531, CD11c/DEC-205, lectins such as Mann-C4-Chol, Maleylated bovine serum albumin (MBSA), O-steroly amylopectin (O-SAP), fibronectin and galactosyl.

Monocytes, macrophages, dendric cells, or their precursor cells can also be edited outside the body of the subject and then introduced to the subject. Such monocytes, macrophages, dendric cells, or their precursor cells may be obtained from a donor subject, or from the target subject himself/herself. Before editing, in some embodiments, the isolated cells can be enriched in monocytes, macrophages, dendritic cells or their precursors. In some embodiments, the cell population includes more than 80% of monocytes, macrophages, dendritic cells or their precursors, and still more preferably more than 90%.

In some embodiments, the isolated precursor cells can be differentiated into monocytes or monocyte-derived cells depending on the need of the human subject. In some embodiments, the cells (e.g., monocytes) isolated may be de-differentiated into precursor cells capable of differentiating to monocytes.

Monocytes are a hematopoietic cell lineage derived from progenitor cells in the bone marrow. Committed myeloid progenitor cells differentiate to form blood monocytes, which circulate in the blood and then enter the tissues to become resident macrophages. A precursor cell, therefore, may be a myeloid progenitor cell or another type of progenitor cell from the bone marrow.

In some embodiments, the cells can be activated, e.g., with interferon gamma (INF-γ) to enhance cytotoxicity. The maturation of the monocytes to macrophages and activation with interferon gamma can be carried out by contacting the INF-γ. Alternatively, the activation can be done by transformation with a recombinant DNA expressing INF-γ.

The edited monocytes, monocyte-derived cells or precursors thereof are then introduced to the subject. In some embodiments, the cells are delivered systemically, e.g., injection into the blood, bone marrow, or a target tissue, without limitation. Moreover, they may be used alone or combined with other pharmaceutical compositions.

### Exon Skipping Inhibitors

Inhibition of exon 3 skipping in the PTS gene in a human cell (or skipping of the corresponding exon in other mammalian cells, in particular primate cells) can also be accomplished or assisted with agents that inhibit the biological activity of the respective splicing factors, such as anyone of SRSF1, SRSF2, SRSF3, SRSF4, SRSF5, SRSF6, SRSF7, SRSF8, SRSF9, SRSF10, SRSF11 and SRSF12. Accordingly, in one embodiment, provided is a method for altering or strengthening the immune system in a mammalian subject in need thereof. In some embodiments, the method entails administering to the subject an agent that inhibits the exon 3 skipping in the 6-pyruvoyltetrahydropterin synthase (PTS) pre-mRNA.

It is contemplated that each of the splicing factors may play a role in the exon 3 skipping. In a preferred embodiment, the agent targets SRSF3, which has a recognition site in intron 2 of the PTS gene, as shown in Example 1. In some embodiments, the agent targets SRSF1 or SRSF9.

Inhibiting the biological activity, or decreasing the biological activity of a splicing factor, can be achieved by decreasing the expression of the splicing factor, and/or interrupting the functioning of the splicing factor protein.

Methods for decreasing the expression of a protein include, without limitation, the use of antisense or RNAi technology. “RNA interference” (RNAi) refers to sequence-specific or gene specific suppression of gene expression (protein synthesis) that is mediated by short interfering RNA (siRNA).

“Short interfering RNA” (siRNA) refers to double-stranded RNA molecules, generally, from about 10 to about 30 nucleotides long that are capable of mediating RNA interference (RNAi). The term siRNA includes short hairpin RNAs (shRNAs). shRNAs comprise a single strand of RNA that forms a stem-loop structure, where the stem consists of the complementary sense and antisense strands that comprise a double-stranded siRNA, and the loop is a linker of varying size. The stem structure of shRNAs generally is from about 10 to about 30 nucleotides long.

In some embodiments, the agent that inhibits the biological activity of a splicing factor is an antibody. As used herein, an “antibody” or “antigen-binding polypeptide” refers to a polypeptide or a polypeptide complex that specifically recognizes and binds to an antigen. An antibody can be a whole antibody and any antigen binding fragment or a single chain thereof. Thus the term “antibody” includes any protein or peptide containing molecule that comprises at least a portion of an immunoglobulin molecule having biological activity of binding to the antigen.

Antibodies, antigen-binding polypeptides, variants, or derivatives thereof of the disclosure include, but are not limited to, polyclonal, monoclonal, multispecific, human, humanized, primatized, or chimeric antibodies, single chain antibodies, epitope-binding fragments, e.g., Fab, Fab′ and F(ab′)₂, Fd, Fvs, single-chain Fvs (scFv), single-chain antibodies, disulfide-linked Fvs (sdFv), fragments comprising either a VK or VH domain, fragments produced by a Fab expression library, and anti-idiotypic (anti-Id) antibodies (including, e.g., anti-Id antibodies to LIGHT antibodies disclosed herein). Immunoglobulin or antibody molecules of the disclosure can be of any type (e.g., IgG, IgE, IgM, IgD, IgA, and IgY), class (e.g., IgGl, IgG2, IgG3, IgG4, IgA1 and IgA2) or subclass of immunoglobulin molecule.

Antibodies disclosed herein may be from any animal origin including birds and mammals. Preferably, the antibodies are human, murine, donkey, rabbit, goat, guinea pig, camel, llama, horse, or chicken antibodies. The antibodies can also have an animal origin and then humanized.

In some embodiments, the antibody has specificity to a splicing factor such as SRSF1, SRSF2, SRSF3, SRSF4, SRSF5, SRSF6, SRSF7, SRSF8, SRSF9, SRSF10, SRSF11 or SRSF12. The antibody may not be bispecific or multi-specific that target two or more of the splicing factors. In one embodiment, the antibody is a monoclonal antibody having specificity to SRSF3.

Small molecule inhibitors to the splicing factors can also be used. The agent can also be a molecule that generally reduce aberrant splicing or promote normal splicing by any other means. Non-limiting examples of the agents include kinetin, epigallocatechin gallate (EGCG), genistein, daidzein, cardiac glycosides (e.g., digoxin), and rectifier of aberrant splicing (RECTAS).

Kinetin (6-furfurylaminopurine) belongs to the family of N6-substituted adenine derivatives known as cytokinins, or plant growth factors, that also includes zeatin, benzyladenine and 2iP. Kinetin has been shown to be able to rescue human mRNA splicing defects and increase the production of normal mRNA (see, e.g., Slaugenhaupt et al., Human Mol. Genet. 2004, 13(4):429-36), likely due to inhibition of splicing factor activities.

Genistein (5,7-Dihydroxy-3-(4-hydroxyphenyl)chromen-4-one) is an isoflavone that has been used as an angiogenesis inhibitor and a phytoestrogen. Daidzein (7-hydroxy-3-(4-hydroxyphenyl)-4H-chromen-4-one) is another naturally occurring isoflavone found in soybeans and other legumes and structurally. Genistein and daidzein and other isoflavones are produced in plants through the phenylpropanoid pathway of secondary metabolism and are used as signal carriers, and defense responses to pathogenic attacks. In humans, recent research has shown that daidzein may be useful for menopausal relief, osteoporosis, blood cholesterol, and lowering the risk of some hormone-related cancers, and heart diseases.

Epigallocatechin gallate (EGCG), also referred to as epigallocatechin-3-gallate, is the ester of epigallocatechin and gallic acid, and is a type of catechin. EGCG, a polyphenol with antioxidant activity, is the most abundant catechin in tea.

It has been shown that genistein and daidzein, optionally together with EGCG, have potent activity in rescuing defective splicing (see, e.g., Anderson, et al., Mol Nutr Food Res. 2012 56(4):570-579).

Cardiac glycosides are a class of organic compounds which are commonly secondary metabolites in several plants such as foxglove plants, that increase the output force of the heart and increase its rate of contractions by acting on the cellular sodium-potassium ATPase pump. The general structure of a cardiac glycoside consists of a steroid molecule attached to a sugar (glycoside) and an R group. The steroid nucleus consists of five fused rings to which other functional groups such as methyl, hydroxyl, and aldehyde groups can be attached to influence the overall molecule's biological activity. Non-limiting examples include digoxin, digitonin, digitoxin, digoxigenin, digitoxigenin, acetyldigitoxin, bufalin, ouabagenin and ouabain.

Researches have shown that cardiac glycosides, including digoxin, digitonin, digitoxin, digoxigenin, digitoxigenin, acetyldigitoxin, bufalin, ouabagenin and ouabain, were able to prevent production of exon-skipped transcripts by inhibiting SRSF3 (Liu et al., FEBS J. 2013 280(15):3632-46).

The name “rectifier of aberrant splicing” or “RECTAS” refers to a compound identified from a drug screen that is able to promote exon recognition and thus reduce aberrant splicing (see, Yoshida et al., Proc Natl Acad Sci USA. 2015;112(9):2764-2769). RECTAS has a chemical name of 2-chloro-N-(furan-2-ylmethyl)-7H-purin-6-amine.

Additional agents can be identified by screening, e.g., using cultured human monocytes as a test model. Correction of the exon skipping can be examined with, for examples, antibodies targeting the wild-type PTPS protein.

The antisense technology, including the use of antisense RNA or antisense oligonucleotides, can be readily used on cis-regulatory elements to inhibit their effect on exon skipping. Such cis-regulatory elements, for instance, can be an exonic splicing silencer or an intron splicing silencer.

### Enhancement of Exon Recognition

The process of exon recognition may require the canonical sequences, e.g., splice sites, polypyrimidine tract and branch site, as well as exonic and intronic cis-acting regulatory elements. Further, whether an exon is recognized also depends on the cellular environment which provides splicing factors. In a cell that does not provide the appropriate cellular environment, agents can be added to enhance/rescue exon recognition.

Exon-specific U1s (ExSpeU1s) have been used to promote recognition of defective exons or normal exons in non-supportive environments. ExSpeU1s are U1 snRNP-like particles that bind by complementarity to intronic regions downstream of the 5′ splice site of skipped exons. They facilitate exon recognition by recruiting the spliceosomal components. ExSpeU1s can be used to correct exon skipping at the consensus 5′ splice site, the polypyrimidine tract or at exonic regulatory elements. ExSpeU1s have successfully restored splicing in various genetic-disorder-genes including hemophilia B, cystic fibrosis, Netherton syndrome and spinal muscular atrophy. Since ExSpeU1s can be designed to bind at non-conserved intronic sequences, they result in very few off target events.

The ExSpeU1 can include a single-stranded U1 snRNA molecule having a portion capable of hybridizing to a target nucleotide sequence on the primary transcript of the target gene of therapeutic interest. The target nucleotide sequence of the U1snRNA molecule may be located in a region of the pre-mRNA comprised between 2 and 50 base pairs downstream of an exon/intron junction site.

The snRNA may be introduced to a target cell by a polynucleotide encoding for U1snRNA molecule. Preferably, the polynucleotide includes a promoter sequence and a polyadenylation signal sequence. The promoter is preferably the endogenous promoter of the gene encoding for human U1 snRNA.

### Nonsense Suppression Agents and Methods

In some embodiments, the normal expression/activity of PTS in a cell wherein the exon 3 (or a corresponding exon in mammalian or primate cell) is skipped can be restored with a nonsense suppression therapy. Nonsense suppression therapies aim at suppressing translation termination at in-frame premature termination codons (PTCs, also known as nonsense mutations) to restore deficient protein function. The exon skipping in PTS leads to the generation of a stop/termination codon, resulting in a shortened inactive transcript. A nonsense suppression therapy, it is contemplated, is able to bring back the majority of the amino acid residues and functional domains of the PTPS protein.

Non-limiting examples of nonsense suppression therapies include readthrough drugs, suppressor tRNAs, PTC pseudouridylation, and inhibition of nonsense-mediated mRNA decay.

Aminoglycosides are a class of antibiotics which have been shown to bind to eukaryotic ribosomes and lead to misincorporation of near-cognate aminoacyl-tRNAs at PTCs. Gentamicin, the aminoglycoside most commonly used for nonsense suppression studies, has been shown to restore functional protein in short-term studies of DMD, CF, nephrogenic diabetes insipidus, hemophilia, retinal degeneration, APC-mediated colon cancer, and mucopolysaccharidosis I-Hurler.

Another example of aminoglycosides is amikacin. In particular, intravenous administration of unilamellar, low-clearance liposomes containing amikacin (MiKasome®) showed good efficacy with reduced toxicity.

Some non-aminoglycoside antibiotics can also suppress PTCs in mammalian cells. Negamycin, a peptide antibiotic that binds to the eukaryotic small ribosomal subunit, suppresses nonsense mutations and restores protein function in the APC gene associated with colon cancer, in the laminin α-2 gene associated with congenital muscular dystrophy, and in the dystrophin gene associated with DMD.

In addition, several macrolide antibiotics, such as spiramycin, josamycin, and tylosin, can suppress APC nonsense mutations and restore APC protein function. In addition, they can reduce tumors and intestinal polyp number and size in mice that carry a nonsense mutation in the APC gene.

Ataluren, previously known as PTC124, is an oxadiazole compound that suppresses termination at PTCs in mammalian cells without affecting translation termination at natural stop codons. Comprehensive preclinical studies showed that PTC124 is safe, has minimal off-target side effects, has no antibacterial activity, and is orally bioavailable.

High-throughput drug screens have identified other compounds that suppress nonsense mutations in the ATM gene that cause ataxia telangiectasia (Du, et al. 2009. J. Exp. Med. 206:2285-97; Du, et al. 2013. Mol. Ther. 21:1653-60). These drugs include RT13, RT14, GJO71, and GJ072 as well as their derivatives. These drugs can restore the expression of full-length, functional ATM protein.

Compounds that inhibit nonsense-mediated mRNA decay (NMD) can also be used. NMD is a pathway to degrade mRNAs containing PTCs. Amlexanox is an NMD inhibitor and also suppresses PTCs.

A “nonsense suppressor tRNA” is a tRNA derivative whose anticodon has been altered to recognize a stop codon, thus allowing the incorporation of an amino acid at the stop codon and a bypass of translation termination. A nonsense suppressor tRNA can be constructed, for instance, by modifying the anticodon of a tRNALys to recognize the UAG nonsense codon instead of the normal AAA (lysine) codon.

“Pseudouridylation” is the isomerization of the ribonucleoside uridine to the 5′-ribosyl isomer pseudouridine (ψ). An H/ACA guide RNA can be used to offer the possibility of site-specific pseudouridylation via sequence homology with any RNA sequence. Pseudouridylation of tRNA molecules results in alternative codon recognition through changes in anticodon loop structure. Because all three nonsense codons contain a uridine (U) in the first position (UAA, UAG, UGA), pseudouridylation of the uridine of stop codons can alter the efficiency of PTC recognition.

### Selection of Patients for Exon Skipping Correction

As provided, restoration of the PTPS protein activity can strengthen the immune system and help prevent and treat diseases such as infectious diseases and cancer. Such restoration can be made at any stage for an individual, in particular those that are susceptible to infections or cancer development. Also, this can be useful for any subjects at times when an improved immune activity is needed.

In some embodiments, the restoration, e.g., through administration of an agent as disclosed herein, can be made to a subject that is at risk of infection or is infected. In some embodiments, the restoration can be made to a subject that is at risk of developing cancer or has cancer. In some embodiments, the restoration can be made to a subject that has oxidative stress intensity. In some embodiments, the restoration can be made to a subject that has increased immune activity.

The immune activity of an individual can be measured with suitable biomarkers. Non-limiting examples of biomarkers include neopterin, C-reactive protein, and procalcitonin. Other examples include interferon gamma, interferon-alpha, and interferon-beta. For instance, higher levels of neopterin and/or interferon gamma, or lower levels of tetrahydrobiopterin and/or nitric oxide indicate that the subject is able to benefit from the disclosed treatment.

Samples that can be used to measure a biomarker include, without limitation, serum, urine, cerebrospinal fluid, synovial fluid, saliva, ascitic fluid, bile, pancreatic juice and gastric juice.

Reference levels of these biomarkers are also known. For instance, over 97% of healthy individuals (both children and adults) have a neopterin concentration in serum below 10 nmol/L. Therefore, when a person has a serum neopterin concentration higher than 10 nmol/L, the person can be selected for restoration of the PTPS protein activity with the disclosed technology. In some embodiments, a subject is selected for the treatment when serum neopterin concentration higher than 10 nmol/L, 20 nmol/L, 30 nmol/L, 50 nmol/L, 100 nmol/L, or 200 nmol/L.

Likewise, a neopterin level in urine, expressed as mol neopterin/mol creatinine, that is higher than 350 μmol neopterin/mol creatinine in subjects younger than 7 or higher than 250 neopterin/mol creatinine in older patients is suggestion that the subject can benefit from restoration of the PTPS protein activity. Similarly, in saliva, a neopterin concentration higher than 3.4 nmol/L, in synovial, a neopterin concentration higher than fluid 9 nmol/L, in the ascitic fluid, a neopterin concentration higher than 26.3 nmol/L, or in bronchoalveolar lavage fluid a neopterin concentration higher than 85.6 nmol/L is indication of increased immune activity.

Reference levels for nitric oxide can also be determined for a subject. In some embodiments, a nitric oxide level (e.g., in the serum or urine) higher than 10 μmol/L can be considered not in need of a treatment. Accordingly, in some embodiments, when a person has a nitric oxide concentration lower than 10 μmol/L, the person can be selected for restoration of the PTPS protein activity with the disclosed technology. In some embodiments, a subject is selected for the treatment when the nitric oxide concentration lower than 0.1 μmol/L, 0.2 μmol/L, 0.5 μmol/L, 1 μmol/L, 2 μmol/L, 3 μmol/L, 4 μmol/L, 5 μmol/L, 10 μmol/L, 15 μmol/L, 20 μmol/L, 30 μmol/L, 40 μmol/L, 50 μmol/L, 60 μmol/L, 70 μmol/L, 80 μmol/L, 90 μmol/L, 100 μmol/L, 150 μmol/L, 200 μmol/L, 300 μmol/L, or 500 μmol/L.

Such biomarkers can also be used to measure the effectiveness of correction/inhibition of exon skipping for the PTS gene. For instance, a decrease in a neopterin level, a decrease of an interferon gamma level, an increase in either BH4, tetrahydrobiopterin, or nitric oxide level can indicate that correcting the exon skipping is effective. In some embodiments, the effectiveness can also be directly measured by detecting mature PTS mRNA with the skipped exon, present in a target cell.

### Therapeutic Compositions and Methods

The genome editing systems, in vitro or ex vivo-treated cells, antisense or RNAi molecules, antibodies or small molecule agents can be prepared as pharmaceutical compositions and used in mammalian, in particular primary and human, subjects in need of them. The pharmaceutical compositions can further include a pharmaceutically acceptable carrier.

The term “pharmaceutically acceptable” means approved by a regulatory agency of the Federal or a state government or listed in the U.S. Pharmacopeia or other generally recognized pharmacopeia for use in animals, and more particularly in humans. Further, a “pharmaceutically acceptable carrier” will generally be a non-toxic solid, semisolid or liquid filler, diluent, encapsulating material or formulation auxiliary of any type.

The term “carrier” refers to a diluent, adjuvant, excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents such as acetates, citrates or phosphates. Antibacterial agents such as benzyl alcohol or methyl parabens; antioxidants such as ascorbic acid or sodium bisulfate; chelating agents such as ethylenediaminetetraacetic acid; and agents for the adjustment of tonicity such as sodium chloride or dextrose are also envisioned. These compositions can take the form of solutions, suspensions, emulsion, tablets, pills, capsules, powders, sustained-release formulations and the like. The composition can be formulated as a suppository, with traditional binders and carriers such as triglycerides. Oral formulation can include standard carriers such as pharmaceutical grades of mannitol, lactose, starch, magnesium stearate, sodium saccharine, cellulose, magnesium carbonate, etc. Examples of suitable pharmaceutical carriers are described in Remington's Pharmaceutical Sciences by E. W. Martin, incorporated herein by reference.

In an embodiment, the composition is formulated in accordance with routine procedures as a pharmaceutical composition adapted for administration to human beings. Typically, compositions for intravenous administration are solutions in sterile isotonic aqueous buffer. Where necessary, the composition may also include a solubilizing agent and a local anesthetic such as lidocaine to ease pain at the site of the injection. Generally, the ingredients are supplied either separately or mixed together in unit dosage form, for example, as a dry lyophilized powder or water free concentrate in a hermetically sealed container such as an ampoule or sachet indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

The pharmaceutical compositions, in some embodiments, are formulated for targeted delivery to the target cells. For instance, the agents may be packaged in liposomes. Liposomes are a commonly used delivery system for monocyte/phagocyte-targeted therapies providing advantages such as low immunogenicity, biocompatibility, cell specificity and drug protection. Parenterally administered liposomes are naturally cleared by the mononuclear phagocytic system (MPS). Liposome drug delivery systems take advantage of the physiological role of these cells to provide specific targeting and enhance delivery efficacy.

Targeting of liposomes to monocytes and monocyte-derived cells can be achieved by modifying lipid composition to control physicochemical properties such as size and charge and by the inclusion of surface ligands including proteins, peptides, antibodies, polysaccharides, glycolipids, glycoproteins, and lectins. Non-limiting examples of such ligands include anionic lipids, muramyl tripeptide (MTP), Arg-Gly-Asp (RGD), antibodies specific to VACM-1, CC52, CC531, CD11c/DEC-205, lectins such as Mann-C4-Chol, Maleylated bovine serum albumin (MBSA), O-steroly amylopectin (O-SAP), fibronectin and galactosyl.

The molecules, cells, systems, and compositions of the present disclosure can be useful for strengthening the immune system in a human subject in need thereof and treat diseases or conditions in the subject. The human subject may be suffering from disease or condition the treatment of which can benefit from the enhanced immune system. Human subjects that are at risk of developing such disease or conditions can also benefit from the treatment.

As used herein, the terms “treat” or “treatment” refer to both therapeutic treatment and prophylactic or preventative measures, wherein the object is to prevent or slow down (lessen) an undesired physiological change or disorder, such as the progression of an infectious disease or cancer. Beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment. Those in need of treatment include those already with the condition or disorder as well as those prone to have the condition or disorder or those in which the condition or disorder is to be prevented.

### Infectious Diseases

In some embodiments, the subject suffers from infection or is at risk of being infected. Infection is the invasion of an organism's body tissues by disease-causing agents, their multiplication, and the reaction of host tissues to these organisms and the toxins they produce. An infection can be caused by infectious agents such as viruses, viroids, prions, bacteria, nematodes such as parasitic roundworms and pinworms, arthropods such as ticks, mites, fleas, and lice, fungi such as ringworm, and other macroparasites such as tapeworms and other helminths. In one aspect, the infectious agent is a bacterium, such as Gram-negative bacterium. In one aspect, the infectious agent is virus, such as DNA viruses, RNA viruses, and reverse transcribing viruses.

Non-limiting examples of viruses include herpesvirus, coronavirus, influenzavirus, filovirus, flavivirus, arenavirus, togavirus, bunyavirus, poxvirus, picornavirus, retrovirus, paramyxovirus, hantavirus, measles, mumps, rubella, viral hepatitis (all types), viral meningitis, human t-lymphotropic virus, crimean-congo hemorrhagic fever, other virus families.

Non-limiting examples of diseases associated with bacterial infection include sepsis/septic shock, pseudomonas, brucella, streptococcal, mycobacterium, salmonella, borrelia, listeria, shigella, and bacterial meningitis.

Non-limiting examples of diseases associated with infections by parasites include plasmodium, leishmaniasis, Schistosoma, Trypanosoma, and giardia.

### Cancers

In some embodiments, the subject has cancer or is at risk of developing cancer. As the method can generally strengthen the immune system of the human subject, it can be helpful for the treatment of a large of cancer types. In some embodiments, the cancer is selected from bladder cancer, non-small cell lung cancer, renal cancer, breast cancer, urethral cancer, colorectal cancer, head and neck cancer, squamous cell cancer, Merkel cell carcinoma, gastrointestinal cancer, stomach cancer, esophageal cancer, ovarian cancer, renal cancer, and small cell lung cancer.

In some embodiments, the subject suffers from a disease selected from leukemia (including acute leukemias (e.g., acute lymphocytic leukemia, acute myelocytic leukemia (including myeloblastic, promyelocytic, myelomonocytic, monocytic, and erythroleukemia)) and chronic leukemias (e.g., chronic myelocytic (granulocytic) leukemia and chronic lymphocytic leukemia)), polycythemia vera, lymphomas (e.g., Hodgkin's disease and non-Hodgkin's disease), multiple myeloma, Waldenstrom's macroglobulinemia, heavy chain disease, and solid tumors including, but not limited to, sarcomas and carcinomas such as fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyo sarcoma, colon carcinoma, pancreatic cancer, breast cancer, thyroid cancer, endometrial cancer, melanoma, prostate cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilm's tumor, cervical cancer, testicular tumor, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, menangioma, melanoma, neuroblastoma and retinoblastoma.

In some embodiments, the method further includes administration of a chemotherapeutic agent. Chemotherapeutic agents that may be administered with the compositions of the disclosure include, but are not limited to, antibiotic derivatives (e.g., doxorubicin, bleomycin, daunorubicin, and dactinomycin); antiestrogens (e.g., tamoxifen); antimetabolites (e.g., fluorouracil, 5-FU, methotrexate, floxuridine, interferon alpha-2b, glutamic acid, plicamycin, mercaptopurine, and 6-thioguanine); cytotoxic agents (e.g., carmustine, BCNU, lomustine, CCNU, cytosine arabinoside, cyclophosphamide, estramustine, hydroxyurea, procarbazine, mitomycin, busulfan, cis-platin, and vincristine sulfate); hormones (e.g., medroxyprogesterone, estramustine phosphate sodium, ethinyl estradiol, estradiol, megestrol acetate, methyltestosterone, diethylstilbestrol diphosphate, chlorotrianisene, and testolactone); nitrogen mustard derivatives (e.g., mephalen, chorambucil, mechlorethamine (nitrogen mustard) and thiotepa); steroids and combinations (e.g., bethamethasone sodium phosphate); and others (e.g., dicarbazine, asparaginase, mitotane, vincristine sulfate, vinblastine sulfate, and etoposide).

In some embodiments, the compositions of the disclosure are administered in combination with cytokines. Cytokines that may be administered with the compositions of the disclosure include, but are not limited to, IL-2, IL-3, IL-4, IL-5, IL-6, IL-7, IL-10, IL-12, IL-13, IL-15, anti-CD40, CD4OL, and TNF-α.

In additional embodiments, the compositions of the disclosure are administered in combination with other therapeutic or prophylactic regimens, such as, for example, radiation therapy.

### Cardiovascular Diseases and Disorders

In some embodiments, the subject has a cardiovascular disease or disorder. Non-limiting examples of cardiovascular diseases and orders include coronary heart diseases, cerebrovascular diseases, peripheral arterial diseases, rheumatic heart diseases, congenital heart diseases, and deep vein thrombosis and pulmonary embolism. Other examples include heart attack and stroke. Improvement of the immune functions in subject can help lower the risk or treat these diseases.

In some embodiments, the subject has a cerebrovascular disease, such as stroke (e.g., ischemic stroke, hemorrhagic stroke, transient ischemic attack (TIA)), carotid stenosis, cerebral aneurysms, vascular malformations, moyamoya diseases, venous angiomas, and vein of galen malformation (VGM).

Additional examples of cardiovascular diseases and conditions include, without limitation, dilated cardiomyopathy, chronic myocarditis, acute rheumatic fever, aortic insufficiencies, mitral insufficiencies, atherosclerosis, arteriosclerosis, acute coronary syndrome, chronic coronary syndrome, coronary artery disease, acute myocardial infarction, unstable angina, non-Q-wave MI, congestive heart failure, left ventricular dysfunction, peripheral arterial disease, critical limb ischemia, acute stroke, acute cerebral ischemia, ischemic stroke, non-ischemic heart failure, pulmonary arterial hypertension, and chronic thromboembolic pulmonary hypertension (CTEPH).

### Autoimmune Diseases and Disorders

In some embodiments, the subject has an autoimmune disease or disorder. In some embodiments, the autoimmune disease or condition to be treated includes one or more of Aicardi-Goutieres syndrome, alopecia areata, ankylosing spondylitis, autoimmune hemolytic anemia, autoimmune hepatitis, dermatomyositis, diabetes (type 1), celiac disease, autoimmune juvenile idiopathic arthritis, Crohn's disease, glomerulonephritis, Graves' disease, Guillain-Barrésyndrome, hemophagocytic lymphohistiocytosis, idiopathic thrombocytopenic purpura, myasthenia gravis, autoimmune myocarditis, multiple sclerosis, pemphigus/pemphigoid, pernicious anemia, peripheral neuropathies, polyarteritis nodosa, polymyositis, primary biliary cirrhosis, psoriasis, rheumatoid arthritis including juvenile rheumatoid arthritis, sarcoidosis, scleroderma/systemic sclerosis, Sjögren's syndrome, systemic lupus erythematosus, autoimmune thyroiditis, Hashimoto's thyroiditis, autoimmune uveitis, ulcerative colitis, vitiligo, and granulomatosis with polyangiitis (Wegener's).

Rheumatoid arthritis (RA) is a long-term autoimmune disorder that primarily affects joints. It typically results in warm, swollen, and painful joints. Pain and stiffness often worsen following rest. Most commonly, the wrist and hands are involved, with the same joints typically involved on both sides of the body. The disease may also affect other parts of the body. While the cause of rheumatoid arthritis is not clear, it is believed to involve a combination of genetic and environmental factors. The underlying mechanism involves the body's immune system attacking the joints. This results in inflammation and thickening of the joint capsule. The goals of treatment are to reduce pain, decrease inflammation, and improve a person's overall functioning. Pain medications, steroids, and NSAIDs are frequently used to help with symptoms. A group of medications called disease-modifying antirheumatic drugs (DMARDs), such as hydroxychloroquine and methotrexate, may be used to try to slow the progression of disease.

Osteoarthritis (OA) is a type of joint disease that results from breakdown of joint cartilage and underlying bone. The most common symptoms are joint pain and stiffness. Initially, symptoms may occur only following exercise, but over time may become constant. Other symptoms may include joint swelling, decreased range of motion, and when the back is affected weakness or numbness of the arms and legs. Causes include previous joint injury, abnormal joint or limb development, and inherited factors. Risk is greater in those who are overweight, have one leg of a different length, and have jobs that result in high levels of joint stress. Osteoarthritis is believed to be caused by mechanical stress on the joint and low grade inflammatory processes. Treatment includes exercise, efforts to decrease joint stress, support groups, and pain medications.

Multiple sclerosis (MS) is a demyelinating disease in which the insulating covers of nerve cells in the brain and spinal cord are damaged. This damage disrupts the ability of parts of the nervous system to communicate, resulting in a range of signs and symptoms, including physical, mental, and sometimes psychiatric problems. Specific symptoms can include double vision, blindness in one eye, muscle weakness, trouble with sensation, or trouble with coordination. While the cause is not clear, the underlying mechanism is thought to be either destruction by the immune system or failure of the myelin-producing cells. There is no known cure for multiple sclerosis. Treatments attempt to improve function after an attack and prevent new attacks.

Asthma is a common long-term inflammatory disease of the airways of the lungs. It is characterized by variable and recurring symptoms, reversible airflow obstruction, and bronchospasm. Symptoms include episodes of wheezing, coughing, chest tightness, and shortness of breath. Asthma is thought to be caused by a combination of genetic and environmental factors. Environmental factors include exposure to air pollution and allergens. Asthma is classified according to the frequency of symptoms, forced expiratory volume in one second (FEV1), and peak expiratory flow rate. It may also be classified as atopic or non-atopic, where atopy refers to a predisposition toward developing a type 1 hypersensitivity reaction. There is no cure for asthma. Symptoms can be prevented by avoiding triggers, such as allergens and irritants, and by the use of inhaled corticosteroids. Long-acting beta agonists (LABA) or antileukotriene agents may be used in addition to inhaled corticosteroids if asthma symptoms remain uncontrolled. Treatment of rapidly worsening symptoms is usually with an inhaled short-acting beta-2 agonist such as salbutamol and corticosteroids taken by mouth. In very severe cases, intravenous corticosteroids, magnesium sulfate, and hospitalization may be required.

Chronic obstructive pulmonary disease (COPD) is a type of obstructive lung disease characterized by long-term poor airflow. COPD can include two main conditions, emphysema and chronic bronchitis. In emphysema, the walls between many of the air sacs are damaged. As a result, the air sacs lose their shape and become floppy. This damage also can destroy the walls of the air sacs, leading to fewer and larger air sacs instead of many tiny ones. If this happens, the amount of gas exchange in the lungs is reduced. In chronic bronchitis, the lining of the airways stays constantly irritated and inflamed, and this causes the lining to swell. Lots of thick mucus forms in the airways, making it hard to breathe. There is no known cure for COPD, but the symptoms are treatable and its progression can be delayed.

### Neurodegenerative Diseases and Disorders

In some embodiments, the subject being treated has as a neurodegenerative disease or disorder. Non-limiting examples of neurodegenerative disease or disorder include multiple sclerosis, Alzheimer's disease, Parkinson's disease, vascular dementia, Huntington's disease, Creutzfeldt-Jakob disease, Down's syndrome, depression, autism spectrum disorder, ADHD/ADD, Amyotrophic lateral sclerosis, Neuromyelitis Optica spectrum disorders,

### Liver, Renal and Other Diseases and Conditions

In some embodiments, the subject has a liver disease or disorder. Non-limiting examples of liver diseases or disorders include hepatitis, cirrhosis, liver cancer, liver failure, ascites, gallstones, hemochromatosis, primary sclerosing cholangitis, and primary biliary cirrhosis. Examples also include nonalcoholic fatty liver disease (NAFLD) and nonalcoholic steatohepatitis (NASH).

In some embodiments, the subject has a renal disease or disorder, such as but not limited to diabetic nephropathy, nephrotic syndrome, glomerulonephritis, chronic kidney disease, and end stage renal disease.

In some embodiments, the subject has a condition associated with transplantation. In one example, the subject has a transplant rejection in the kidney, the heart, the liver, or the pancreas. In another example, the subject has a graft versus host disease (GVHD). In another example, the subject has rejection related to blood transfusion.

Additional diseases and conditions that can be suitably treated with the instant technology include, without limitation, sarcoidosis, periodontitis, acute pancreatitis, intrauterine infection, burns, aging, macrophage activation syndrome, hip fractures, pneumoconiosis, acute-on-chronic liver failure, systemic inflammatory response syndrome, obesity, and diabetes mellitus.

### EXAMPLES

The following examples are included to demonstrate specific embodiments of the disclosure. It should be appreciated by those of skill in the art that the techniques disclosed in the examples which follow represent techniques to function well in the practice of the disclosure, and thus can be considered to constitute specific modes for its practice. However, those of skill in the art should, in light of the present disclosure, appreciate that many changes can be made in the specific embodiments which are disclosed and still obtain a like or similar result without departing from the spirit and scope of the disclosure.

### Example 1: Identification of Cis-Regulatory Element Affecting Exon Skipping

This example examines the genomic sequence of the human 6-pyruvoyltetrahydropterin synthase (PTS) for cis-regulatory elements related to the regulation of skipping of exon 3.

The human genomic sequence of PTS can be found in in GenBank ID: 5805 (NG 008743.1: nucleotides 5001 to 12609), which is reproduced in Table 1 below.

TABLE 1
Genomic Sequence of Human PTS (exons are underlined)
SEQ ID NO: 1

```
      1 GCGCAGCCGC GGTGGGAGGA GGCACCGGCC GCGCGGCGGG AGGAGGTGCC GGCCGAGCAC
    61 CGCAGACAGC GCCGGGAAGA TGAGCACGGA AGGTGGTGGC CGTCGCTGCC AGGCACAAGT Exon 1
  121 GTCCCGCCGC ATCTCCTTCA GCGCGAGCCA CCGATTGTAC AGGTAGGGTG TGCACACAGG
  181 TACAGCGGCG GGCGGTGGGC GCCGGGCCCC GGAACGTCAC CGGGGCGGGG CCGGCGGGCG
  241 TGCTGACGTC GGGCCCGGGA GGGGCGCGGG GGCTGCTGGG GCGACGCGCG CTGGTCGGCT
  301 TCGTGGGGCT TCGGACGGCC TCCAGCATCC TGATGGGGGC TGGAGTGTCC CCAGCCCTGG
  361 AGGGGTGGGG GAGCTTGATG GTTAACGGAG CCGACTGCGG AGGGCGATGG CCCACGTCTG
  421 GGTGCGGGGC CCACACCCGG TTCTGCGACT CGAAGAAACG TCTCTGCCCC TAGGAGTCCC
  481 TTGGTGTAGA CCACAGGGTT GCTGTGAAAC TCAGCAGTGT TAAACTCGCC TATGTTACAC
  541 AGTACCTGGC GCGGCGTAGA CACTCAGTAA ATGTTAGCTA ATCTTGTATG CCAGGAGCAT
  601 TGGCCACCAT ATCTAATGTA ATTACAAGGC AAATTTTAGC CCCTTTTTTA CCAAAGACTG
  661 GATGAACCCA GTCCCTGGTG GTCTAGTGGC TAGGAGAAAA AAAAAAAAAA ATGAGCCCAG
  721 ACGTTCAGTT TCCGAAGCAG CATAGTTGGG ATTTGAATCC CGATCATACA TACTGCTTGC
  781 ATGCACTGAC GAGTTGATTT TGAGGAATTT TACCACGCAC TAACCAGCTC CACCCTCTGG
  841 CCCTCCGTAT CGTCGTCTGT GAAATGAGGA TGACCATCGC TATCTAACAG GATTCATCCC
  901 TCCATTCGCT AAACCAGGTT TTGTTTCGTG CTTGTCAGGG CTTGTGCTAG GTAACGGATG
  961 CATCAGGGAT CTTGTATGGG GTTTCCAATC TGGCCTATTT TGTGGAGGGT ACACTGAGAA
1021 AATTTTGTGC AAAAGACTTT AGCACTCTAA ATACAAGAAG TGGCTGAGCA GGAGAAAGTG
1081 GGGGTGTGTT ATTCCATTTT GCATTGCCAC AAAGGAATAC TGAGACTGGT ATTTTATAAA
1141 GAAAAGAGGT TTATTTGGTT CTGCAGGCTG TACAAGAAGC ATGGCACCAG CATCTGCTTC
1201 TGTTGAGGCC TCAGGGACCT TCCACTCATG GTGGAAGGCG AAGGGCGAGC AAGCAGGCAA
1261 CTCACAGGGC AAGTGAGGGA GCAAGAGAGA GGAGAGGAGG TGCCAGGCTT TTTTTAACAA
1321 CCAGCTCGCC CATTGACTAA TAGAGCGAGA ACTCACTCAT TGCTTCAGGG AGGGCACCAA
1381 GCCATTCATA AGGGATCCAC TTCCATGAGC CAAACATCTC CCACTAGGCC CCATCTCCAA
1441 CACTGGGGAT CACATTTCAA CATGACAATT GGAGGGGACA AACATCCAAA CTACATTGGG
1501 GGGAATAATA AAGAGGATCT CAAAAAAAAG GGGTTGTATA ATAAGCTTAA ACACCTGAGG
1561 GTGGTAAGGT CTCATAGACG ATGCGAAATT GAATGGATGG GTATAAGTAT AAACATTAAG
1621 CAATTTGTTT TCTGTCTGAG ACGACAGCCA AAGATTGTAA TTTTAAAAAT TCACCTGCTT
1681 GGAATATTCC TCAACAGATT GGTAAAGTTC TCATGAGGCA GTAGTTGAGA CATAGTATAA
1741 AGCAAGATTG CTAAATAGGA GACTTACCAG GTCATGTAAT TTTTTTAAAT TATTTTTTCT
1801 GGATGAGTAG TTTAGCTTCT GAGAGAAATG AGATAGTAAG CCACTTTGCG GATCACCATC
1861 TATGTTTATC ATTATATGCT GTTATTTCAT TCAGCACTTG CCTGTTACAT TGTAGGCACT
1921 CAGTAATGTT GAATTGAAAA GTGGAAGGCC CATGAGCAGA TCAGTTGCTG TGGAACAAGG
1981 GGGTTGAAGT TAAGGTTTGT TTGTGCTAAT TTGTATGGTA CAATCTTCTA ATTAGGGAAT
2041 ATGCCATGGT TTGTGACGTA TACGTAAGTA ATAAAATCAA CATGATTTCT GACTCTCCCT
2101 TTGGTGAGCT AAAGTAATAA ATTGGGAAAC TTTTCAAAGA TCAGTACAAA TAATAAATAT
2161 AAGGAACAGA GAAGGGGGTT TGAATGTGAT ACTTGTGTCA TGCTGACTTT TTTTTTTTTT
2221 TTTGGTCAGT AAATTTCTAA GTGATGAAGA AAACTTGAAA CTGTTTGGGA AATGCAACAA Exon 2
2281 TCCAAATGGC CATGGGCACA ATTATAAAGG TGAGAGAAAA ACTGATGACA TTTCAGCCCT
2341 TCAATAAGGA TGAAAGAGTA TTCAGCAAAT GTAGACATAA AGAATGGGAA AACTTACGGA
2401 CACAGTGTGA ATGCTTTGAG CCTTGAATGA GAAATTAAAT GGGAGTTCAG AATGAAAGGA
2461 TCTGTTGTCT TGGTTGGGTG TGTGTTAAGT TTTACCTTGC AATGTCAACT CTTACAAACA
2521 GTCCAAAACA ATGAATGGTT TAAAGCATTT TTCTTTGTCC TAGAGTACAC AACAAGTTTC
2581 TTTCTGATTA GGTATGGCCA AGGCTAATGT TAAAATTAGA TTTTATTCTT TGAGTAAATA
2641 ATGACTGACA AAAGGGGAAT GTTGCCTAGG AATGTAATGT ATGGCAGAAA AATAGGTTTG
2701 GTCTAAAGCT TTGCCAGCAG TGTTCGAAGA AAGTAGGTAA TTTGTTCAAG GATAAGAGAC
2761 TTGATTCTTT GAGGTACAGC CTGCCTTAAA AAACAGGTAC ATAAGTCGTA GAGTATAAAA
2821 CAGTATGTGG TGTGGTGGCA TAAAAAAAAA CAGACAAGGT GTCAGGAGAT TAGGATTACT
2881 GGTTTAGCTA CAGCCTCTTC CTTAATCTAT GGTATCTCAG TCTCAGTGTA CTCACTTACA
2941 TGTAATATCT AATGGTATTT GTACAGAACT AAAATACATA AATTCATAAA AGTTATTAAT
3001 GTGTAAAGCA CTGATAAAGT TTTTTTTTGT TGTTGTTGTT TTTTTTTTTG AGATGGAGTT
3061 CCACTCTTTT TGCCCAGGCT GGAATGGTGT GATCTCCCGT CACTACAACC TCCACCTCCC
3121 AGGTTCGAGC GATTCTCCAG CCTCAGCCTC CCGAGTAGCT GAGATTACAG GCGCCCGCCA
3181 CTATGCCTGG CTAATTTTTG TATTTTTAGT AGAGATGGGG TTTCACCATG TTGGCCAGGC
3241 TGCTCTCAAA CTCCAGACCT CAGGTGATCT GCCCGCCTCG GCCTCCCGAA GTGCTGGGAT
3301 TACAGGCTTG AGCCACCACG CCTGGCCAAG ATTTTTCAAA TGCTTCTCAC AAGCACTTTT
3361 TAATTTACAA AGCATTACTT TAGACTTTGT ATACATTCAT GTGAGTAAGG GGACAAGAAT
3421 TTAAGGCCCA GTAGTATTGC TTGTTCAGAA AGGTACTGCT CAGTATTGGA CCAGCTTGCT
3481 GTGTAGCTGG ACACCTGAAA GACATTTTCT ACTTCTACAG GCAGAGACTG AGCAGCATCT
3541 TGATGCTCCC TAGTGCATTA ACATAAGCAG AATTTACACC CTTTTCAGCC TTGGCCGAGT
3601 GCCTCTGCTT AGCCAACATT CCTACTAAGC TCCTTTGTCT CGATTGTGTC TTGCTTTGGT
3661 TGCTAAAAAA AAAATCTTAA CTAAAATAAC AGATGTTTTG GGGTAAATAT TTAAGTATAG
3721 CTTTTGGGGA CAGATCTAAT AATTTATGTT GCCAACTTGT GCTTGTATGT TGCTAACTTG
3781 TGCTTGGATG TTGATCTGTT GAAAGTCATG CTGTTTTTTT TGTATTTTGT TTTCTTTCCA
3841 TAGTTGTGGT GACAGTACAT GGAGAGGTAT GTGCAGAAAA TATTTGTGTG GTTTTTGCAG Exon 3
3901 ATTGCTGGGC TCTCTTTCAG CCAGTGTGGT GGATTCTGTG TTGAAAACTG TTCCAGTCAG
3961 TATTGCTTCA TTGTTGGCCC TTGTATATGT GTGTGTGGTG AGCTGCCGCC ATTCCAGGCC
4021 TCTCCTCTAC CAAAGTGTTG TCTTTAATAT GCTGTATGTG GACAGGTAGA AGGGCTATAC
4081 AATGAAAAGG ATGCTTGAAG TACATGGTGC TTCCATGCTG AGGTCAATGA TTATCTCTGG
4141 GATGAAGGCA AATGTGCAAA TGTGGGCACA GTCTCTGCAC ATTGTACTGC CTTTAATAAT
4201 TTGCCAGCCG TTTAATATGG AGAGCCTATC ACAGTAATAT TCACCTTTGT TTATTCTTTA
4261 GATTGACCCT GCTACGGGAA TGGTTATGAA TCTGGCTGAT CTCAAAAAAT ATATGGAGGT Exon 4
4321 AATGGCATGT TGGGTGCTTA TTATGTGCTA TTCCCTAACT GTAATATTTG GTGGCCCCCT
4381 ATCTACCTCC CCAACCAGTT ATCTCCTAAG GTTCCATGAC TTTGTGAATA GAACTGGATG
4441 TGGGTGTTGG GGAATAGTTG GAAGAACTGC TCGCATGGCC TCTAAAGGTG TTTTGGTGAC
4501 TTAACGGAAA TTAGTCACAG TTGTGTTACA GCGATGGGAG GAAATATAAC ATGCTCACCT
4561 TGTATTCCTT GTTGGAATAT CCTGATTTCC TTCAGTTAAC AAATTCTCCA TTTCTTTTAA
4621 GACATAGCTT TCTCCTTGAG ACTTACTGTC TTTCTTTTTT TTTTTTTTTT TAAATAGGCA
4681 CAGAACCAAT GCAAACTTTT CTTTTTGGTA GAGGTAGGGG TCTCACTATG TTGTTCAGGC
4741 TGGTCTCAGA CACCTCCTGC CTTGACCTCC CAAAGTGCTG GAATTACAGG CATGAGCTAC
4801 TGCTCCTGGC CTTCTCCTTG GAGCTTTCTC TTCCCTCTCT GAATATTCCT TGTACCTTTG
4861 ATCATAGATT TAGACACCAC ACTGTGTTGT TCATTCTAGC TTTATTATAA ACTCACCAGA
4921 GCAGTACCTC ATAGAGATAA CACACAAGGT GTTCAATGAA GAACTTAACT GATTGTGTCA
4981 TTATAGAATT CTTGCACTGT AAGAGACTTT GGAGATTATT CAGACCAACT TCTTCAACCC
5041 ATAGATTAAG ATTGAGAGAT GCTGAACAGT TAACTGACTT GCCCCAGGTC ATATGGCTAG
5101 TCATTTGCAA AGCTGGGAGT AGATTTCTTT CCTTCCAGCT GTTTTATTAT GCTGTCATTT
5161 CACTTGCAGC TTAGGTTTGT TCTGAGATTT GACATTTGCT TGATTAAAAT GATGTTATTA
5221 GTAATGATTA CTTCTTTCAC ATTACTTATT CATATATTCA ACAAATTTCT CTACTTCGCA
5281 TCAGTTTTTG AGCTAAGTGT ACAAAGATGA ATGAGATGTA GCCCACAGAG TGTATACATT
5341 ACTTTTTCTA GGAGGTGAAG AATATTATTA AATAACTGTT GATTTAATAA ATGAGTAGAA
5401 GAATGAGTGA ATGAAGAAAC AGCACTATGC TCAAAATACT TTAGGAAAGT TATACAGATA
5461 GGGCCAGCAT GCATTTATGG GGCAGGGGTT GGGGGGCAGG CGGAGAGAGA GAGAGAGGGA
5521 GACAGAGACA GGAGAAGAGG AGGGGGAGGG GAGGACAGGG GAGGAGAGGG GAGGGGAAAG
5581 AAAGAAAAAG AAAAAGAGAA AAGAAAAGAA AAGAAAGAAA AGAGACAAGA CAGCAACATA
5641 AAAGGCCTCA ACATATGACT AAATACTGGA GGAGGGCTTC TCTATGCTGT TTTCTGCTTT
5701 TGTATGCCTG AAGGACACCA CACACTTTCA TCAGTAAAAG TGGCTTATCA TGGCATGTAG
5761 AGTAAAGTTA GAGCTCTACC GTGGGGGGAG GATTGCTTGA GTCCAGGAGT TCAAGGCTGC
5821 AGTGAGCTAT GATTATGCCA CTTGCACTCC AGCCTGGATG ACAGAGACCC TGTCTCTAAA
5881 AACAAAACAA AAGAAAACAA AAAGCCCTGC ATAAGAGGCT TTAATGGAGA AATTGGGACT
5941 GGAACTGGGA TTGAAGATGA AGCAAGGATG CCCGTTTTCT TGTTATTCAT GTGGAACATC
6001 CCTTCATTGG ATATATTTCA ATAACTGTTT GATCTAGGAC TTACTCTTTT TGTATGATTC
6061 TAGTTTTTCT CAAGAAATGA TTGCTGTTAC TGTTATTAAT GTACCCTTCT TCAAACTACG
6121 TCCTATGGAA AATTACATTT TTTTTTCACA GATGCTTAGA TTTTTTGCAA TAGCTAAAAG
6181 CAAAAACCTA GAAAGAATGA GGCAATTTAA TCCTTGCTGA TGATGAGGAA AAACATTATG
6241 TTAATATACA CGAGCATTAG GGAGTAATAG TCTTTACATA TATATATTTT TCCTTCTATG
6301 AGCCCAAGAT CATTCTTAAA TTATTCCTCA TAGCAAAAAT TTAGAACTAC TTACATAATT
6361 TCAGTCTAGG TCTGCTTTAT AAAATGTAGT AGTCACTGGA TCTTTGTCCT TATGATATTG
6421 TGGCATTGTT CCGTAGGTGT GACAGTGTTA TGGAAAAATA TTTGTGCTAA CTGTTTGGAC
6481 AGGATTCACA AAGTGTTAAA CTGAATTCTG AATTACAATG GCTTGTGTTG CGAGAAAGCT
6541 CCAGGAATTC TCTGCTTTTT GGTTTCTCAC CTCTTTTTAT TCAGTAACAA GGATTCCTAA
6601 TCATTACCGA CAGCTGGGCC TGACTTTATT TTACAACTTG AAATTTTGTA AAGTTGCCTT
6661 GTAAGACTCA AATCTAGTAC TTACAAATAT TTAGTTAGTG GCTAAGTGAT AAGGTGAGGT
6721 TTAGAGGCAT AAGTGGAACA ATTTGGAATT TGAGTCGTAA ATGGAGTCAA TGATATTTTC
6781 CCTTGGTTTT GTCTCTAGGA GGCGATTATG CAGCCCCTTG ATCATAAGAA TCTGGATATG Exon 5
6841 GATGTGCCAT ACTTTGCAGA TGTGGTGAGG TGGGTGGCAC TGTATCTTGC CTTATGTGGA
6901 TTGTAAAACA AGAATTGATT TGAATACTTT GATTGTTGTG TGATTTCTGA AGTTTTAATT
6961 TAATGAAATC TTTCGAAACT AGAATTTCTA TTTTCTGTAA ATATTAAACA TGAAATTTTA
7021 TTGTTTGCAT TTTGAATTTT TTTTGTTTTT GTTTTTTTTT CTTATAGCAC GACTGAAAAT Exon 6
7081 GTAGCTGTTT ATATCTGGGA CAACCTCCAG AAAGTTCTTC CTGTAGGAGT TCTTTATAAA
7141 GTAAAAGTAT ACGAAACTGA CAATAATATT GTGGTTTATA AAGGAGAATA GCTATTGGGG
7201 TTAGCATTGC ACAAAGCCCA GTTTCTTTCT GTGTTTGAAA AAGATTTTGA TCCCCTTGGA
7261 ATATTAAGAG GTCAACACGT GATTGTTGTA CGTACACATT GTGCTCTGGA GTGCCTATTT
7321 ATTGAAATCA TTGTAAGACC TGTTATAAAT TTAAGTCTAT TTAAAACTAA ACTTGTAATA
7381 TACATCCTGA AAATCATTTA GAGAGTCTTT TATTTATAAA TTAAAAATCA CTTCATTTTC
7441 ACAAAATGTT TTGGTGTGGG ATTATTTGAA AGCAAAAGAA ATCTAATTTT GTTTTCTCCA
7501 TTACCTCATT TTAGTATTAA TTTTTACTTG GTATAATATA CATGGTTAAA ATGCTTATGT
7561 GACTTCGAGT AGGTGAATCT TAAAGAAATA AAATTCAAGT GACCACAAA
```

The sequence was examined to identify cis-regulatory element motifs targeted by protein factors involved in splicing, in particular splicing factor, arginine/serine-rich proteins such as SRSF1-12 (see Table 2).

TABLE 2
Splicing Factors

| Splicing factor | Aliases |
|---|---|
| SRSF1 | SFRS1, ASF, SF2, SRp30a |
| SRSF2 | SFRS2, SC35, PR264, SRp30b |
| SRSF3 | SFRS3, SRp20 |
| SRSF4 | SFRS4, SRp75 |
| SRSF5 | SFRS5, SRp40, HRS |
| SRSF6 | SFRS6, SRp55, B52 |
| SRSF7 | SFRS7, 9G8 |
| SRSF8 | SFRS2B, SRp46 |
| SRSF9 | SFRS9, SRp30c |
| SRSF10 | SFRS13A, TASR1, SRp38, SRrp40 |
| SRSF11 | SFRS11, p54, SRp54 |
| SRSF12 | SFRS13B, SRrp35 |

The search was focused within the two introns surrounding exon 3 that is skipped in human monocytes and macrophages. The most significant findings are listed in Table 3.

TABLE 3
Identified cis-regulatory elements

| | | Potential sites in introns 2 (2330:3843) and 3 |
|---|---|---|
| Cis element | Pattern | (3867:4261) (location relative to SEQ ID NO: 1) |
| SRSF3 | CUCKUCY | CTCTTCC (2895-2901) |
| SRSF3 | WCWWC | TCAAC (2505-2509); ACAAC (2569-2573); TCTTC |
| | | (2896-2900); ACTAC (3102-3106); TCTAC (3508- |
| | | 3512); TCTAC (3508-3512); TCTAC (3514-3518); |
| | | TCTAC (4026-4030) |
| SRSF1 | UGRWGVH | TGGTGGC (2833-2839); TGATGCT (3541-3547); |
| | | TGGTGGA (3927-3933); TGGTGCT (4105-4111); |
| | | TGAAGGC (4143-4149) |
| SRSF2 | GRYYMCYR | GATTACTG (2874-2881) |
| SRSF2 | UGCYGYY | TGCTGTT (3809-3815) |
| | | TGCCGCC (4004-4010) |
| SRSF5 | YYWCWSG | TTACTGG (2876-2882); TTACAGG (3165-3171); |
| | | CCTCAGG (3258-3264); TTACAGG (3300-3306); |
| | | CTACAGG (3515-3521); TCTCTGG (4134-4140) |
| SRSF6 | YRCRKM | TACGGA (2395-2400); TACAGC (2775-2780); |
| | | TACATA (2798-2803); TACAGC (2889-2894); |
| | | TACAGA (2962-2967); TACATA (2975-2980); |
| | | TGCAGA (3872-3877); TGCAGA (3896-3901) |

| IUPAC nucleotide code | Base |
|---|---|
| A | Adenine |
| C | Cytosine |
| G | Guanine |
| T (or U) | Thymine (or Uracil) |
| R | A or G |
| Y | C or T |
| S | G or C |
| W | A or T |
| K | G or T |
| M | A or C |
| B | C or G or T |
| D | A or G or T |
| H | A or C or T |
| V | A or C or G |
| N | any base |
| . or − | gap |

It is contemplated that each of these identified cis-regulatory elements may play a role in regulating exon 3 skipping in the PTS gene. In particular, the sole binding site of SRSF3, CTCTTCC (2895-2901 of SEQ ID NO:1), within intron 2 may play a significant role in SRSF3-mediated exon 3 skipping.

Unless otherwise defined, all technical and scientific terms used herein have the same meaning as commonly understood by one of ordinary skill in the art to which this invention belongs.

The inventions illustratively described herein may suitably be practiced in the absence of any element or elements, limitation or limitations, not specifically disclosed herein. Thus, for example, the terms “comprising”, “including,” “containing”, etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed.

All publications, patent applications, patents, and other references mentioned herein are expressly incorporated by reference in their entirety, to the same extent as if each were incorporated by reference individually. In case of conflict, the present specification, including definitions, will control.

1. A method for inhibiting or correcting the skipping of exon 3 in the 6-pyruvoyltetrahydropterin synthase (PTS) mRNA or promoting recognition of the exon 3 in a cell, comprising editing the PTS genomic sequence of pre-mRNA, or contacting the cell with an agent that inhibits exon skipping or promotes exon recognition or inclusion.

2. A method for modulating the immune system in a mammalian subject in need thereof, comprising editing the 6-pyruvoyltetrahydropterin synthase (PTS) genomic sequence or pre-mRNA in a monocyte, a macrophage, a dendritic cell or a precursor cell thereof in the mammalian subject, wherein the editing inhibits skipping of exon 3 in the PTS mRNA during splicing.

3. The method of claim 2, wherein the editing alters a recognition site of a splicing factor.

4. The method of claim 3, wherein the recognition site is located within intron 2 or intron 3.

5. The method of claim 3, wherein the splicing factor is selected from the group consisting of SRSF1, SRSF2, SRSF3, SRSF4, SRSF5, SRSF6, SRSF7, SRSF8, SRSF9, SRSF10, SRSF11 and SRSF12.

6. The method of claim 5, wherein the recognition site is selected from Table 3.

7. A method for modulating the immune system in a mammalian subject in need thereof, comprising administering to the subject an agent that inhibits exon 3 skipping or promotes exon 3 recognition in the 6-pyruvoyltetrahydropterin synthase (PTS) pre-mRNA in a monocyte, a macrophage, a dendritic cell or a precursor cell thereof in the subject, or administering a nonsense suppression agent to a tissue in the subject that produces or circulates monocytes, macrophages, dendritic cells or precursor cells thereof

8. The method of claim 7, wherein the agent inhibits the biological activity of a splicing factor selected from the group consisting of SRSF1, SRSF2, SRSF3, SRSF4, SRSF5, SRSF6, SRSF7, SRSF8, SRSF9, SRSF10, SRSF11 and SRSF12.

9. The method of claim 8, wherein the splicing factor is SRSF3.

10. The method of claim 8, wherein the agent is an antisense oligonucleotide or RNA, a siRNA, or a shRNA that inhibits the expression of the splicing factor, or is an antibody having specificity to the splicing factor.

11. The method of claim 7, wherein the agent is a small molecule agent selected from the group consisting of kinetin, epigallocatechin gallate (EGCG), genistein, daidzein, a cardiac glycoside, and rectifier of aberrant splicing (RECTAS).

12. The method of claim 11, wherein the cardiac glycoside is selected from the group consisting of digoxin digitonin, digitoxin, digoxigenin, digitoxigenin, acetyldigitoxin, bufalin, ouabagenin and ouabain.

13. The method of claim 7, wherein the agent is a U1snRNA that recognizes exon 3 in the PTS pre-mRNA.

14. The method of claim 7, wherein the nonsense suppression agent is selected from the group consisting of a stop codon readthrough drug, a suppressor tRNA, a stop codon pseudouridylation agent, and a nonsense-mediated mRNA decay (NMD) inhibitor.

15. The method of claim 7, wherein the nonsense suppression agent is selected from the group consisting of ataluren, an aminoglycoside, gentamicin, amikacin, negamycin, spiramycin, josamycin, tylosin and amlexanox.

16. The method of claim 7, wherein the tissue is bone marrow.

17. The method of claim 7, wherein the administration is targeted at the monocyte, macrophage, dendritic cell or the precursor cell thereof.

18. The method of claim 7, wherein the subject suffers from an infectious disease or condition, or cancer or is at risk of developing an infectious disease or condition, or cancer.

19. The method of claim 7, wherein the subject has increased immune activity as compared to a healthy individual.

20. The method of claim 19, wherein the increased immune activity is measured with a biomarker from a sample selected from the group consisting of serum, urine, cerebrospinal fluid, synovial fluid, saliva, ascitic fluid, bile, pancreatic juice and gastric juice.


SEQUENCE LISTING (1 sequence)

SEQ ID NO: 1 - DNA, 7609 bp, Homo sapiens
gcgcagccgcggtgggaggaggcaccggccgcgcggcgggaggaggtgccggccgagcaccgcagacagcgccgggaagatgagcacggaaggtggtggccgtcgctgccaggcacaagtgtcccgccgcatctccttcagcgcgagccaccgattgtacaggtagggtgtgcacacaggtacagcggcgggcggtgggcgccgggccccggaacgtcaccggggcggggccggcgggcgtgctgacgtcgggcccgggaggggcgcgggggctgctggggcgacgcgcgctggtcggcttcgtggggcttcggacggcctccagcatcctgatgggggctggagtgtccccagccctggaggggtgggggagcttgatggttaacggagccgactgcggagggcgatggcccacgtctgggtgcggggcccacacccggttctgcgactcgaagaaacgtctctgcccctaggagtcccttggtgtagaccacagggttgctgtgaaactcagcagtgttaaactcgcctatgttacacagtacctggcgcggcgtagacactcagtaaatgttagctaatcttgtatgccaggagcattggccaccatatctaatgtaattacaaggcaaattttagccccttttttaccaaagactggatgaacccagtccctggtggtctagtggctaggagaaaaaaaaaaaaaaatgagcccagacgttcagtttccgaagcagcatagttgggatttgaatcccgatcatacatactgcttgcatgcactgacgagttgattttgaggaattttaccacgcactaaccagctccaccctctggccctccgtatcgtcgtctgtgaaatgaggatgaccatcgctatctaacaggattcatccctccattcgctaaaccaggttttgtttcgtgcttgtcagggcttgtgctaggtaacggatgcatcagggatcttgtatggggtttccaatctggcctattttgtggagggtacactgagaaaattttgtgcaaaagactttagcactctaaatacaagaagtggctgagcaggagaaagtgggggtgtgttattccattttgcattgccacaaaggaatactgagactggtattttataaagaaaagaggtttatttggttctgcaggctgtacaagaagcatggcaccagcatctgcttctgttgaggcctcagggaccttccactcatggtggaaggcgaagggcgagcaagcaggcaactcacagggcaagtgagggagcaagagagaggagaggaggtgccaggctttttttaacaaccagctcgcccattgactaatagagcgagaactcactcattgcttcagggagggcaccaagccattcataagggatccacttccatgagccaaacatctcccactaggccccatctccaacactggggatcacatttcaacatgacaattggaggggacaaacatccaaactacattggggggaataataaagaggatctcaaaaaaaaggggttgtataataagcttaaacacctgagggtggtaaggtctcatagacgatgcgaaattgaatggatgggtataagtataaacattaagcaatttgttttctgtctgagacgacagccaaagattgtaattttaaaaattcacctgcttggaatattcctcaacagattggtaaagttctcatgaggcagtagttgagacatagtataaagcaagattgctaaataggagacttaccaggtcatgtaatttttttaaattattttttctggatgagtagtttagcttctgagagaaatgagatagtaagccactttgcggatcaccatctatgtttatcattatatgctgttatttcattcagcacttgcctgttacattgtaggcactcagtaatgttgaattgaaaagtggaaggcccatgagcagatcagttgctgtggaacaagggggttgaagttaaggtttgtttgtgctaatttgtatggtacaatcttctaattagggaatatgccatggtttgtgacgtatacgtaagtaataaaatcaacatgatttctgactctccctttggtgagctaaagtaataaattgggaaacttttcaaagatcagtacaaataataaatataaggaacagagaagggggtttgaatgtgatacttgtgtcatgctgacttttttttttttttttggtcagtaaatttctaagtgatgaagaaaacttgaaactgtttgggaaatgcaacaatccaaatggccatgggcacaattataaaggtgagagaaaaactgatgacatttcagcccttcaataaggatgaaagagtattcagcaaatgtagacataaagaatgggaaaacttacggacacagtgtgaatgctttgagccttgaatgagaaattaaatgggagttcagaatgaaaggatctgttgtcttggttgggtgtgtgttaagttttaccttgcaatgtcaactcttacaaacagtccaaaacaatgaatggtttaaagcatttttctttgtcctagagtacacaacaagtttctttctgattaggtatggccaaggctaatgttaaaattagattttattctttgagtaaataatgactgacaaaaggggaatgttgcctaggaatgtaatgtatggcagaaaaataggtttggtctaaagctttgccagcagtgttcgaagaaagtaggtaatttgttcaaggataagagacttgattctttgaggtacagcctgccttaaaaaacaggtacataagtcgtagagtataaaacagtatgtggtgtggtggcataaaaaaaaacagacaaggtgtcaggagattaggattactggtttagctacagcctcttccttaatctatggtatctcagtctcagtgtactcacttacatgtaatatctaatggtatttgtacagaactaaaatacataaattcataaaagttattaatgtgtaaagcactgataaagtttttttttgttgttgttgtttttttttttgagatggagttccactctttttgcccaggctggaatggtgtgatctcccgtcactacaacctccacctcccaggttcgagcgattctccagcctcagcctcccgagtagctgagattacaggcgcccgccactatgcctggctaatttttgtatttttagtagagatggggtttcaccatgttggccaggctgctctcaaactccagacctcaggtgatctgcccgcctcggcctcccgaagtgctgggattacaggcttgagccaccacgcctggccaagatttttcaaatgcttctcacaagcactttttaatttacaaagcattactttagactttgtatacattcatgtgagtaaggggacaagaatttaaggcccagtagtattgcttgttcagaaaggtactgctcagtattggaccagcttgctgtgtagctggacacctgaaagacattttctacttctacaggcagagactgagcagcatcttgatgctccctagtgcattaacataagcagaatttacacccttttcagccttggccgagtgcctctgcttagccaacattcctactaagctcctttgtctcgattgtgtcttgctttggttgctaaaaaaaaaatcttaactaaaataacagatgttttggggtaaatatttaagtatagcttttggggacagatctaataatttatgttgccaacttgtgcttgtatgttgctaacttgtgcttggatgttgatctgttgaaagtcatgctgttttttttgtattttgttttctttccatagttgtggtgacagtacatggagaggtatgtgcagaaaatatttgtgtggtttttgcagattgctgggctctctttcagccagtgtggtggattctgtgttgaaaactgttccagtcagtattgcttcattgttggcccttgtatatgtgtgtgtggtgagctgccgccattccaggcctctcctctaccaaagtgttgtctttaatatgctgtatgtggacaggtagaagggctatacaatgaaaaggatgcttgaagtacatggtgcttccatgctgaggtcaatgattatctctgggatgaaggcaaatgtgcaaatgtgggcacagtctctgcacattgtactgcctttaataatttgccagccgtttaatatggagagcctatcacagtaatattcacctttgtttattctttagattgaccctgctacgggaatggttatgaatctggctgatctcaaaaaatatatggaggtaatggcatgttgggtgcttattatgtgctattccctaactgtaatatttggtggccccctatctacctccccaaccagttatctcctaaggttccatgactttgtgaatagaactggatgtgggtgttggggaatagttggaagaactgctcgcatggcctctaaaggtgttttggtgacttaacggaaattagtcacagttgtgttacagcgatgggaggaaatataacatgctcaccttgtattccttgttggaatatcctgatttccttcagttaacaaattctccatttcttttaagacatagctttctccttgagacttactgtctttctttttttttttttttttaaataggcacagaaccaatgcaaacttttctttttggtagaggtaggggtctcactatgttgttcaggctggtctcagacacctcctgccttgacctcccaaagtgctggaattacaggcatgagctactgctcctggccttctccttggagctttctcttccctctctgaatattccttgtacctttgatcatagatttagacaccacactgtgttgttcattctagctttattataaactcaccagagcagtacctcatagagataacacacaaggtgttcaatgaagaacttaactgattgtgtcattatagaattcttgcactgtaagagactttggagattattcagaccaacttcttcaacccatagattaagattgagagatgctgaacagttaactgacttgccccaggtcatatggctagtcatttgcaaagctgggagtagatttctttccttccagctgttttattatgctgtcatttcacttgcagcttaggtttgttctgagatttgacatttgcttgattaaaatgatgttattagtaatgattacttctttcacattacttattcatatattcaacaaatttctctacttcgcatcagtttttgagctaagtgtacaaagatgaatgagatgtagcccacagagtgtatacattactttttctaggaggtgaagaatattattaaataactgttgatttaataaatgagtagaagaatgagtgaatgaagaaacagcactatgctcaaaatactttaggaaagttatacagatagggccagcatgcatttatggggcaggggttggggggcaggcggagagagagagagagggagacagagacaggagaagaggagggggaggggaggacaggggaggagaggggaggggaaagaaagaaaaagaaaaagagaaaagaaaagaaaagaaagaaaagagacaagacagcaacataaaaggcctcaacatatgactaaatactggaggagggcttctctatgctgttttctgcttttgtatgcctgaaggacaccacacactttcatcagtaaaagtggcttatcatggcatgtagagtaaagttagagctctaccgtggggggaggattgcttgagtccaggagttcaaggctgcagtgagctatgattatgccacttgcactccagcctggatgacagagaccctgtctctaaaaacaaaacaaaagaaaacaaaaagccctgcataagaggctttaatggagaaattgggactggaactgggattgaagatgaagcaaggatgcccgttttcttgttattcatgtggaacatcccttcattggatatatttcaataactgtttgatctaggacttactctttttgtatgattctagtttttctcaagaaatgattgctgttactgttattaatgtacccttcttcaaactacgtcctatggaaaattacatttttttttcacagatgcttagattttttgcaatagctaaaagcaaaaacctagaaagaatgaggcaatttaatccttgctgatgatgaggaaaaacattatgttaatatacacgagcattagggagtaatagtctttacatatatatatttttccttctatgagcccaagatcattcttaaattattcctcatagcaaaaatttagaactacttacataatttcagtctaggtctgctttataaaatgtagtagtcactggatctttgtccttatgatattgtggcattgttccgtaggtgtgacagtgttatggaaaaatatttgtgctaactgtttggacaggattcacaaagtgttaaactgaattctgaattacaatggcttgtgttgcgagaaagctccaggaattctctgctttttggtttctcacctctttttattcagtaacaaggattcctaatcattaccgacagctgggcctgactttattttacaacttgaaattttgtaaagttgccttgtaagactcaaatctagtacttacaaatatttagttagtggctaagtgataaggtgaggtttagaggcataagtggaacaatttggaatttgagtcgtaaatggagtcaatgatattttcccttggttttgtctctaggaggcgattatgcagccccttgatcataagaatctggatatggatgtgccatactttgcagatgtggtgaggtgggtggcactgtatcttgccttatgtggattgtaaaacaagaattgatttgaatactttgattgttgtgtgatttctgaagttttaatttaatgaaatctttcgaaactagaatttctattttctgtaaatattaaacatgaaattttattgtttgcattttgaattttttttgtttttgtttttttttcttatagcacgactgaaaatgtagctgtttatatctgggacaacctccagaaagttcttcctgtaggagttctttataaagtaaaagtatacgaaactgacaataatattgtggtttataaaggagaatagctattggggttagcattgcacaaagcccagtttctttctgtgtttgaaaaagattttgatccccttggaatattaagaggtcaacacgtgattgttgtacgtacacattgtgctctggagtgcctatttattgaaatcattgtaagacctgttataaatttaagtctatttaaaactaaacttgtaatatacatcctgaaaatcatttagagagtcttttatttataaattaaaaatcacttcattttcacaaaatgttttggtgtgggattatttgaaagcaaaagaaatctaattttgttttctccattacctcattttagtattaatttttacttggtataatatacatggttaaaatgcttatgtgacttcgagtaggtgaatcttaaagaaataaaattcaagtgaccacaaa